(19) United States
(12) Patent Application Publication
(10) Pub. No.: US 2026/0201465 A1
(43) Pub. Date: Jul. 16, 2026

(54) METHOD FOR CONSTRUCTING SPATIAL TRANSCRIPTOME LIBRARY

(71) Applicant: STOmics Tech Co., Ltd., Shenzhen (CN)

(72) Inventors: Zhenzhen ZHU, Shenzhen (CN); Jing GUO, Shenzhen (CN); Yasheng LIU, Shenzhen (CN); Shanshan GUO, Shenzhen (CN); Sha LIAO, Shenzhen (CN); Ao CHEN, Shenzhen (CN); Wenwei ZHANG, Shenzhen (CN)

(73) Assignee: STOmics Tech Co., Ltd., Shenzhen (CN)

(21) Appl. No.: 19/487,676

(22) PCT Filed: Nov. 10, 2023
(86) PCT No.: PCT/CN2023/131048

§ 371 Date: Nov. 25, 2025

(51) Int. Cl. C12Q 1/6869; C12N 15/10; C12Q 1/6806; C12Q 1/6855; C12Q 1/686

(52) U.S. Cl. CPC C12Q 1/6869; C12N 15/1065; C12N 15/1096; C12Q 1/6806; C12Q 1/6855; C12Q 1/686

(57) ABSTRACT

Disclosed in the present invention is a method for constructing a sequencing library. The method comprises: (1) performing reverse transcription treatment on a mRNA to obtain a reverse transcription product without the need of adding a template switch oligo, the 3′ end of the mRNA containing a poly-A sequence, the mRNA being connected to a chip, the chip being connected to a probe containing a poly-T sequence, and the connection being realized by means of the complementary pairing of the Poly-A sequence at the 3′ end of the mRNA and the poly-T sequence on the chip; and (2) performing fragmentation on the reverse transcription product and ligating a first adapter to same so as to obtain a fragmentated and adapter-ligated product, the fragmentated and adapter-ligated product being connected to the chip; and (3) releasing the fragmentated and adapter-ligated product from the chip, so as to obtain a sequencing library.

### FIELD

The present disclosure belongs to the technical field of gene sequencing, and in particular, the present disclosure relates to a method for constructing a spatial transcriptome library.

### BACKGROUND

In multicellular organisms, gene expression of individual cells occurs in a strictly specific temporal and spatial order, that is, gene expression exhibits both temporal specificity and spatial specificity. Temporal specificity can be analyzed by sampling specimens at different time points and applying single-cell transcriptome sequencing technologies to resolve cell types and gene expression patterns along the temporal dimension. In contrast, spatial specificity information is relatively more difficult to obtain. Therefore, by characterizing transcriptomes in situ within tissues, spatial transcriptome technologies provide important biological information for cell typing, characterization of cell states, and cell-cell interactions.

However, conventional spatial transcriptome technologies rely on template switch oligos (TSOs) for amplification. TSOs have low addition efficiency and require mRNAs to possess a 5′ cap structure for addition. Degraded mRNAs may lack such a cap structure, and therefore cannot add TSOs for subsequent PCR amplification.

Accordingly, there is an urgent need to develop a spatial transcriptome library construction method that does not rely on TSOs for amplification.

### SUMMARY

The present disclosure is intended to address, at least in part, one of the technical problems in the related art. To this end, one objective of the present disclosure is to provide a method for constructing a spatial transcriptome library.

The present disclosure has been accomplished based on the inventors' following findings:

As shown in FIG. 1, conventional spatial transcriptome technologies typically involve the following steps:
1. The poly-A sequence at the 3′ end of mRNA hybridizes with a poly-T probe on a spatial chip. Reverse transcription is carried out using the mRNA as a template. During this process, the reverse transcriptase adds three additional cytosine (C) bases at the position corresponding to the 5′ cap structure. At this point, a template switch oligo (TSO) containing three guanine (G) bases is introduced, which serves as a template for further reverse transcription to synthesize the complementary sequence of the TSO.
2. The cDNA obtained from reverse transcription in step 1 is released from the spatial chip.
3. PCR amplification is performed using adapter 2 and the TSO sequence as primers, thereby generating double-stranded cDNA.
4. The double-stranded cDNA is fragmented using a transposase, and adapter 1 is added.
5. PCR amplification is performed using primers containing adapter 2 sequences and primers containing adapter 1 sequences to obtain a library comprising positional barcodes and mRNA sequences, which can be subjected to sequencing analysis.

However, the above-described spatial transcriptome technology has the following disadvantages: 1) It relies on TSOs for amplification. TSOs exhibit low addition efficiency, and addition is only possible when the mRNA contains a 5′ cap structure. However, degraded mRNAs may lack the cap structure, thereby preventing the addition of TSOs and subsequent PCR amplification. (2) It requires a first round of PCR amplification of cDNA, followed by fragmentation of the amplified product using a transposase, simultaneous addition of adapter 1 to the fragmented product, and a second round of PCR amplification of the fragmented products with adapters at both ends. A high number of cycles in these two rounds of PCR amplification may increase the number of duplicate sequences in the data. (3) Because the cDNA must be released and then subjected to two rounds of PCR amplification, the overall workflow is lengthy.

In contrast, the method of the present disclosure enables the construction of a sequencing library without the addition of a TSO, and without requiring PCR amplification of the cDNA prior to fragmentation with a transposase and the addition of adapter 1. This method shortens the workflow and time of sequencing library construction, reduces the cost, enables the capture of mRNAs that lack a 5′ cap structure due to degradation in a sample, and reduces the occurrence of duplicate sequences in the sequencing library.

In view of the foregoing, in a first aspect, the present disclosure provides a method for constructing a sequencing library. According to embodiments of the present disclosure, the method includes:

Step (1): Subjecting mRNA to reverse transcription without adding a template switch oligo, thereby obtaining a reverse transcription product. The 3′ end of the mRNA comprises a poly-A sequence. The mRNA is attached to a chip to which a probe comprising a poly-T sequence is attached by complementary pairing between the poly-A sequence at the 3′ end of the mRNA and the poly-T sequence on the chip.

Step (2): Subjecting the reverse transcription product to fragmentation and addition of a first adaptor, thereby obtaining a fragmented adapter-ligated product. The fragmented adapter-ligated product remains attached to the chip.

Step (3): Releasing the fragmented adapter-ligated product from the chip, thereby obtaining the sequencing library.

The method of the present disclosure can reduce the cost by shortening the workflow and the time of constructing the sequencing library, enable capture of mRNAs that lack a 5′ cap structure due to degradation in a sample, and reduce duplicate sequences in the sequencing library.

According to an embodiment of the present disclosure, the above method may further include at least one of the following technical features:
According to an embodiment of the present disclosure, the reverse transcription product includes a cDNA-mRNA hybrid strand. The cDNA is a first strand cDNA.

According to an embodiment of the present disclosure, step (1) further includes, subsequent to the reverse transcription, synthesizing a second strand cDNA. The synthesis of the second strand cDNA is carried out using a random primer and using the first strand cDNA as a template.

According to an embodiment of the present disclosure, the synthesis of the second strand cDNA is carried out using a polymerase, and the polymerase is at least one selected from Bst polymerase, Taq DNA polymerase, Klenow fragment, T4 DNA polymerase, reverse transcriptase having DNA-dependent polymerase activity, and DNA polymerase I.

According to an embodiment of the present disclosure, the polymerase is Bst polymerase.

According to an embodiment of the present disclosure, the synthesis of the second strand cDNA is carried out under a condition of 65° C. to 70° C. for 20 to 60 minutes.

According to an embodiment of the present disclosure, the probe is of a plurality of types, and each type of probe includes a unique positional barcode sequence, which has a one-to-one correspondence with the location of the type of probe on the chip.

According to an embodiment of the present disclosure, the probe further includes a second adapter sequence. The 5′ end of the second adapter sequence is attached to the chip, the 3′ end of the second adapter sequence is linked to the 5′ end of the positional barcode sequence, and the 3′ end of the positional barcode sequence is linked to the poly-T sequence.

According to an embodiment of the present disclosure, the probe further includes a unique molecular identifier sequence. The 5′ end of the second adapter sequence is linked to the chip, the 3′ end of the second adapter sequence is linked to the 5′ end of the positional barcode sequence, the 3′ end of the positional barcode sequence is linked to the 5′ end of the unique molecular identifier sequence, and the 3′ end of the unique molecular identifier sequence is linked to the poly-T sequence.

According to an embodiment of the present disclosure, the mRNA is derived from a tissue sample or a single-cell sample.

According to an embodiment of the present disclosure, the method further includes, prior to step (1), contacting the tissue sample or single-cell sample with the chip; and permeabilizing the tissue sample or single-cell sample to release mRNA from the tissue sample or single-cell sample.

According to an embodiment of the present disclosure, in step (2), the reverse transcription product is fragmented using a transposase or a fragmentase.

According to an embodiment of the present disclosure, the transposase is Tn5 transposase.

According to an embodiment of the present disclosure, in step (3), the fragmented adapter-ligated product is released from the chip using a lysis solution.

According to an embodiment of the present disclosure, the lysis solution is an alkaline solution or formamide.

According to an embodiment of the present disclosure, the lysis solution is a strong alkaline solution.

According to an embodiment of the present disclosure, the lysis solution is KOH.

According to an embodiment of the present disclosure, step (3) further includes, subsequent to said releasing the fragmented adapter-ligated product from the chip, subjecting the fragmented adapter-ligated product to PCR amplification, thereby obtaining the sequencing library.

In a second aspect, the present disclosure provides a method for sequencing transcriptome. According to an embodiment of the present disclosure, the method for sequencing transcriptome includes: sequencing the sequencing library obtained by the method of the first aspect to obtain sequence information of the sequencing library. The method for sequencing transcriptome according to the embodiments of the present disclosure offers advantages such as fewer library construction steps, shorter processing time, and lower cost, while also enabling the capture of mRNAs that lack a 5′ cap structure due to degradation in a sample.

In a third aspect, the present disclosure provides a method for sequencing spatial transcriptome. According to an embodiment of the present disclosure, the method for sequencing spatial transcriptome includes: constructing a sequencing library using the method of the first aspect; sequencing the sequencing library; and obtaining spatial transcriptome information of the sample to be detected based on the sequencing results. The method for sequencing spatial transcriptome according to embodiments of the present disclosure offers advantages such as fewer library construction steps, shorter processing time, and lower cost, while also enabling mRNAs that lack a 5′ cap structure due to degradation in a sample, as well as reducing duplicate sequences in the sequencing library.

Additional aspects and advantages of the present disclosure will be set forth in part in the following description, and in part will be apparent from the description, or may be learned through practice of the present disclosure.

### BRIEF DESCRIPTION OF DRAWINGS

The foregoing and/or additional aspects and advantages of the present disclosure will become apparent and more readily appreciated from the following description of the embodiments, taken in conjunction with the accompanying drawings.

FIG. 1 is a schematic diagram illustrating the construction of a sequencing library using conventional spatial transcriptome technology.

FIG. 2 is a schematic diagram illustrating the construction of a sequencing library according to one embodiment of the present disclosure.

FIG. 3 shows the results of library fragment quality control using a Bioanalyzer 2100 in Example 1 of the present disclosure.

### DETAILED DESCRIPTION

Hereinafter, embodiments of the present disclosure are described in detail. The embodiments described below are exemplary and are only used to illustrate the present disclosure and are not to be construed as limiting the present disclosure.

It should be noted that the terms “first” and “second” are used for descriptive purposes only and are not to be construed as indicating or implying relative importance or as implicitly indicating the number of technical features indicated. Thus, a feature defined as “first” or “second” may explicitly or implicitly include one or more of the features. Further, in the description of the present disclosure, unless otherwise specified, the meaning of “a plurality of” means two or more than two.

As used herein, the terms “include” or “comprise” are open-ended expressions, i.e., the inclusion of the stated features of the present disclosure does not exclude other features.

As used herein, the terms “optionally”, “optional”, or “option” generally mean that the subsequently described event or circumstance may but may not occur, and that the description includes instances where the event or circumstance occurs and instances in which it does not.

The present disclosure provides a method for constructing a sequencing library, a method for sequencing transcriptome, and a method for sequencing spatial transcriptome, each of which will be described in detail below.

### Sequencing Library and Method for Constructing Sequencing Library

In a first aspect, the present disclosure provides a method for constructing a sequencing library. According to an embodiment of the present disclosure, the method includes:

Step (1): Subjecting mRNA to reverse transcription without adding a template switch oligo, thereby obtaining a reverse transcription product. The 3′ end of the mRNA includes a poly-A sequence. The mRNA is attached to a chip to which a probe comprising a poly-T sequence is attached by complementary pairing between the poly-A sequence at the 3′ end of the mRNA and the poly-T sequence on the chip.

Step (2): Subjecting the reverse transcription product to fragmentation and addition of a first adaptor, thereby obtaining a fragmented adapter-ligated product. The fragmented adapter-ligated product remains attached to the chip.

Step (3): Releasing the fragmented adapter-ligated product from the chip, followed by PCR amplification, thereby obtaining the sequencing library.

The method according to the embodiments of the present disclosure does not require the addition of a template switch oligonucleotide (TSO), thereby avoiding the problems in conventional spatial transcriptome technologies associated with low TSO addition efficiency and poor quality of the constructed sequencing libraries. In addition, in the present disclosure, the reverse transcription product on the chip is directly fragmented and ligated with the first adapter, avoiding the cumbersome steps in conventional spatial transcriptome technologies that require PCR amplification and purification of the reverse transcription product prior to fragmentation and adapter ligation. Therefore, the method of the present disclosure can reduce the cost by shortening the workflow and the time of constructing the sequencing library, enable capture of mRNAs that lack a 5′ cap structure due to degradation in a sample, and reduce duplicate sequences in the sequencing library.

According to an embodiment of the present disclosure, the above method may further include at least one of the following technical features:

According to an embodiment of the present disclosure, the reverse transcription product includes a cDNA-mRNA hybrid strand. The cDNA is a first strand cDNA. Accordingly, the product obtained in step (1) may be directly subjected to steps (2) to (4), thereby obtaining the sequencing library, which significantly shortens the workflow and time for constructing the sequencing library, reduces construction costs, enables the capture of mRNAs that lack a 5′ cap structure due to degradation in a sample, and reduces duplicate sequences in the sequencing library.

It should be noted that the cDNA-mRNA hybrid strand is obtained by performing reverse transcription using the mRNA as a template.

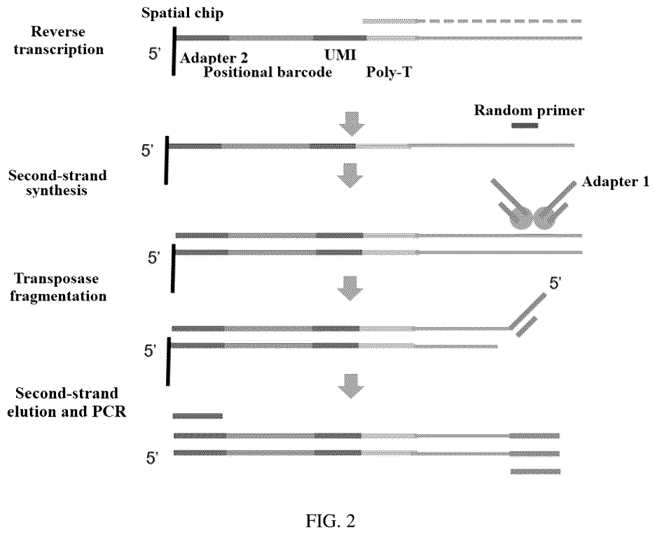

According to an embodiment of the present disclosure, as shown in FIG. 2, step (1) further includes, subsequent to the reverse transcription, synthesizing a second strand cDNA. The synthesis of the second strand cDNA is carried out using a random primer and using the first strand cDNA as a template. As a result, the second strand cDNA contains the information of the mRNA. The second strand cDNA may then be subjected to subsequent library construction and sequencing, followed by data analysis to obtain a spatial transcriptomic map of the sample to be detected or a spatial distribution map of a nucleotide sequence of interest within the host. Moreover, this method eliminates the need for TSO addition, thereby greatly shortening the workflow and time for library construction, reducing costs for library construction, enabling capture of mRNAs that lack a 5′ cap structure due to degradation in a sample, and reducing duplicate sequences in the sequencing library.

In an optional embodiment of the present disclosure, step (1) further includes, subsequent to the reverse transcription and prior to the synthesis of the second strand cDNA, removing the mRNA strand in the cDNA-mRNA hybrid strand. The removal can be carried out using any conventional method known in the art, provided that the mRNA strand is removed, thereby allowing synthesis of the second strand cDNA using the first strand cDNA as a template.

By way of example, removal of the mRNA strand may be performed by alkaline hydrolysis or enzymatic digestion.

In an optional embodiment of the present disclosure, the nucleotide sequence of the random primer is represented as N (4-10), where N represents any one of A, T, C, or G.

By way of example, the nucleotide sequence of the random primer may be represented as NNNNNNNN, where N represents any one of A, T, C, or G.

According to an embodiment of the present disclosure, the synthesis of the second strand cDNA is carried out using a polymerase, and the polymerase is at least one selected from Bst polymerase, Taq DNA polymerase, Klenow fragment, T4 DNA polymerase, DNA polymerase I, and reverse transcriptase having DNA-dependent polymerase activity. By way of example, the reverse transcriptase having DNA-dependent polymerase activity may be Maxima H Minus Reverse Transcriptase. Accordingly, use of the above polymerases can improve the synthesis efficiency of the second strand cDNA, thereby enhancing the quality of the sequencing library.

According to an embodiment of the present disclosure, the synthesis of the second strand cDNA is performed in a mixture including the random primer and the polymerase.

According to an embodiment of the present disclosure, the polymerase is Bst polymerase. Accordingly, synthesis efficiency of the second strand cDNA can be further improved, thereby enhancing the quality of the sequencing library.

In some optional embodiments of the present disclosure, the mixture per 100 μL contains 200 U Bst polymerase, 0.1 mmol dNTP mix, 1 μg random primer, 5 U RNase H, and Bst buffer.

According to an embodiment of the present disclosure, the synthesis of the second strand cDNA is carried out under a condition of 65° C. to 70° C. for 20 to 60 minutes. Accordingly, the synthesis of the second strand cDNA can be achieved.

According to an embodiment of the present disclosure, the probe is of a plurality of types, and each type of probe includes a unique positional barcode sequence, which has a one-to-one correspondence with the location of the type of probe on the chip.

According to an embodiment of the present disclosure, the probe further includes a second adapter sequence. The 5′ end of the second adapter sequence is attached to the chip, the 3′ end of the second adapter sequence is linked to the 5′ end of the positional barcode sequence, and the 3′ end of the positional barcode sequence is linked to the poly-T sequence.

It should be noted that the term “positional barcode” refers to a tag that marks the spatial position on a chip by attaching different nucleic acid sequences to the chip. By way of example, the positional barcode may be a spatial encoding sequence (also referred to as a “barcode”). A sequencing primer for the spatial encoding sequence is used to sequence the spatial encoding sequence by primer hybridization and extension, and each spatial encoding sequence is then spatially located based on imaging, thereby obtaining spatial coordinates. The specific sequences of the spatial encoding sequence and the sequencing primer for the spatial encoding sequence are not limited, provided that spatial localization can be achieved.

As used herein, the terms “first adapter sequence” and “adapter 1” are synonymous; and the terms “second adapter sequence” and “adapter 2” are synonymous. Reference can be made to FIG. 2 for adapter 1 and adapter 2.

According to an embodiment of the present disclosure, the probe further includes a unique molecular identifier sequence. The 5′ end of the second adapter sequence is attached to the chip, the 3′ end of the second adapter sequence is linked to the 5′ end of the positional barcode sequence, the 3′ end of the positional barcode sequence is linked to the 5′ end of the unique molecular identifier sequence, and the 3′ end of the unique molecular identifier sequence is linked to the poly-T sequence.

As used herein, the term “unique molecular identifier” (UMI) refers to a short randomized or specific nucleotide sequence. By adding a UMI to each DNA molecule prior to PCR amplification, the sequencing library constructed therefrom allows, after sequencing, the read sequences with different UMIs to represent different DNA molecules, whereas the read sequences with the same UMI represent PCR duplicates of the same original DNA molecule, thereby enabling discrimination among different mRNA molecules of origin.

In some optional embodiments of the present disclosure, the 5′ end of the second adapter is chemically bonded to the chip.

By way of example, the 5′ end of the second adapter may be modified with DBCO (dibenzocyclooctyne), and the chip may be modified with azide. The 5′ end of the second adapter and the chip are thereby linked through a click chemistry reaction between DBCO and azide.

According to an embodiment of the present disclosure, the mRNA is derived from a tissue sample. That is, the mRNA is provided in the form of a tissue sample.

According to an embodiment of the present disclosure, the mRNA is derived from a single-cell sample. That is, the mRNA is provided in the form of a single-cell suspension.

According to an embodiment of the present disclosure, the method further includes, prior to step (1), contacting the tissue sample or single-cell sample with the chip; and permeabilizing the tissue sample or single-cell sample to release mRNA from the tissue sample or single-cell sample. As a result, spatial localization of the tissue sample or single-cell sample can be achieved for spatial transcriptome sequencing.

According to an embodiment of the present disclosure, in step (2), the reverse transcription product is fragmented using a transposase or a fragmentase.

According to an embodiment of the present disclosure, the transposase is Tn5 transposase.

According to an embodiment of the present disclosure, in step (3), the fragmented adapter-ligated product is released from the chip using a lysis solution.

According to an embodiment of the present disclosure, the lysis solution is an alkaline solution or formamide.

According to an embodiment of the present disclosure, the lysis solution is a strong alkaline solution.

According to an embodiment of the present disclosure, the lysis solution is a KOH solution.

In an optional embodiment of the present disclosure, the concentration of the KOH solution is 0.05 M to 0.2 M.

According to an embodiment of the present disclosure, step (3) further includes, subsequent to said releasing the fragmented adapter-ligated product from the chip, subjecting the fragmented adapter-ligated product to PCR amplification, thereby obtaining the sequencing library. By performing PCR amplification on the fragmented adapter-ligated product, the sequencing read number can be appropriately increased, and the captured mRNA information content can be enriched, thereby rendering the results of spatial transcriptome sequencing analysis more accurate.

### Method for Sequencing Transcriptome and Method for Sequencing Spatial Transcriptome

In a second aspect, the present disclosure provides a method for sequencing transcriptome. According to an embodiment of the present disclosure, the method for sequencing transcriptome includes: sequencing the sequencing library obtained by the method of the first aspect to obtain sequence information of the sequencing library. The method for sequencing transcriptome according to the embodiments of the present disclosure offers advantages such as fewer library construction steps, shorter processing time, and lower cost, while also enabling the capture of mRNAs that lack a 5′ cap structure due to degradation in a sample, as well as reducing duplicate sequences in the sequencing library.

In a third aspect, the present disclosure provides a method for sequencing spatial transcriptome. According to an embodiment of the present disclosure, the method for sequencing spatial transcriptome includes: constructing a sequencing library using the method of the first aspect; sequencing the sequencing library; and obtaining spatial transcriptome information of the sample to be detected based on the sequencing results. The method for sequencing spatial transcriptome according to embodiments of the present disclosure offers advantages such as fewer library construction steps, shorter processing time, and lower cost, while also enabling the capture of mRNAs that lack a 5′ cap structure due to degradation in a sample, as well as reducing duplicate sequences in the sequencing library.

The embodiments of the present disclosure are described in detail below. The embodiments described herein are exemplary and are provided solely for the purpose of explaining the present disclosure, and should not be construed as limiting the present disclosure. Unless otherwise specified, the specific technologies or conditions employed in the embodiments may be selected according to techniques or conditions described in the literature in the field, or according to the instructions provided with commercial products. Unless otherwise specified, reagents or instruments used in the embodiments are conventional products commercially available.

In the following embodiments of the present disclosure, the chips, permeabilization reagents, and reverse transcription reagents were obtained from the BGI Stereo-seq Transcriptome Reagent Kit (Cat. No.: 201ST114), and the fragmentation and PCR reagents were obtained from the BGI Stereo-seq Library Preparation Kit (Cat. No.: 101KL114).

### EXAMPLES

### 1. Tissue Mounting and Fixation

1.1 A Stereo-seq chip T-carrier was removed from a vacuum-sealed aluminum foil bag, the identifier on the back of the chip was recorded, and the chip was equilibrated at room temperature for 1 minute.
1.2 A PCR instrument was pre-set to 37° C. with the heated lid set to 42° C., and a PCR adapter was placed inside for temperature equilibration.
1.3 Methanol was precooled by adding a sufficient amount into a slide box or a 50 mL centrifuge tube to ensure that the methanol could completely immerse the chip, closing the lid and precooling the methanol at −20° C. for 5-30 minutes.
1.4 Tissue sections of 10 μm thickness were cut using a cryostat and flattened, and the tissue was mounted onto the front side of the chip.
1.5 The tissue mounted carrier was immediately placed onto the PCR adapter and incubated at 37° C. for 5 minutes to dry.
1.6 The dried chip was immediately immersed into the precooled methanol at −20° C. for 30 minutes to fix the tissue, ensuring that the chip was completely submerged.
1.7 Once fixed, the slide box or 50 mL centrifuge tube was transferred to a fume hood.
1.8 The carrier was removed from the slide box or 50 mL centrifuge tube and excess methanol on the back and edges of the carrier was blotted dry with dust-free paper.
1.9 The carrier was placed vertically on a slide staining rack and air-dried in the fume hood for 4-6 minutes to allow complete evaporation of methanol.
1.10 After methanol evaporation, the carrier was transferred to the workbench. A gasket and clamp were assembled into a holder, and the chip was secured onto the holder to form a handheld device.

### 2. Tissue Permeabilization

2.1 2 mL of 0.01 N HCl was prepared in advance. A 1× permeabilization working solution was prepared as follows: PR Enzyme was dissolved with 1 mL of freshly prepared 0.01 N HCl, mixed thoroughly by pipetting, and then 10 μL of 10× permeabilization storage solution was diluted into 100 μL with 0.01 N HCl to yield the 1× permeabilization working solution.
2.2 Two PCR instruments (or one PCR instrument and one metal bath) were pre-set to 37° C. with the heated lid at 42° C.
2.3 The handheld device prepared in step 1 was placed on the PCR adapter, the lid of the PCR instrument was closed, and the device was equilibrated at 37° C. for 3 minutes. At the same time, the 1× permeabilization working solution was placed in the PCR instrument or metal bath and equilibrated at 37° C. for 3 minutes.
2.4 After equilibration, the 1× permeabilization working solution was added to a corner of the chip at 100 μL per chip, and the handheld device was sealed with sealing film. The lid of the PCR instrument was then closed.
2.5 The permeabilization reaction was performed at 37° C. for 11 minutes.

### 3. Reverse Transcription

3.1 The RT Reagent and RT Additive were thawed in advance, and kept on ice after thawing.
3.2 An RT reaction mixture (RT Mix) was prepared according to Table 1 and kept on ice.

TABLE 1
RT Reaction Mixture (RT Mix)

| Component | Volume per reaction | |
|---|---|---|
| RT Reagent | 80 | μL |
| RT Additive | 5 | μL |
| RI | 5 | μL |
| Water | 5 | μL |
| ReverseT Enzyme | 5 | μL |
| Total | 100 | μL |

3.3 Another PCR instrument was set to 42° C. with the heated lid at 47° C., and another PCR adapter was placed to equilibrate at this temperature.
3.4 The handheld device obtained from step 2 after permeabilization was removed from the PCR instrument.
3.5 The handheld device was gently tilted at an angle less than 20°, and the permeabilization solution was aspirated from one corner of the chip using a pipette.
3.6 A volume of 100 μL of PR Rinse Buffer solution was added.
3.7 The handheld device was again gently tilted at an angle less than 20°, and the PR Rinse Buffer solution was aspirated from one corner of the chip using a pipette, leaving the chip surface moist.
3.8 The prepared RT Mix was pipette-mixed thoroughly, briefly centrifuged, and then added from one corner of the chip to ensure complete and uniform coverage of the chip surface.
3.9 The handheld device was sealed with sealing film, placed on the PCR adapter of the PCR instrument at 42° C. The lid of the PCR instrument was then closed for 3 hours of reaction.
   4. Second Strand cDNA Synthesis
4.1 A PCR instrument was set to 65° C. with the heated lid at 70° C., and a PCR adapter was placed to equilibrate at this temperature.
4.2 Near the completion of the RT reaction, a reaction mixture for synthesizing second strand cDNA was prepared according to Table 2, vortexed to mix thoroughly, and briefly centrifuged for use.

TABLE 2
Reaction Mixture for Synthesizing Second Strand cDNA

| Component | Volume per reaction | |
|---|---|---|
| 10x Bst buffer (Yeasen Biotechnology, 14402ES92) | 10 | μL |
| 25 mM dNTP | 6 | μL |
| 0.1 μg/μL random primer | 10 | μL |
| 5 U/μL RNaseH (Yeasen Biotechnology, | 1 | μL |
| 14402ES92) | | |
| 40 U/μL Bst polymerase (Yeasen Biotechnology, | 5 | μL |
| 14402ES92) | | |
| Water | 68 | μL |
| Total | 100 | μL |

The sequence of the random primer was NNNNNNNN, where N represents any one of A, T, C, or G.
4.3 Upon completion of the RT reaction, the handheld device from step 3 was removed from the PCR instrument. The device was gently tilted at an angle less than 20°, and the RT Mix solution was aspirated from one corner of the chip using a pipette.
4.4 The prepared reaction mixture for synthesizing second strand cDNA was pipette-mixed thoroughly, briefly centrifuged, and then added from one corner of the chip to ensure complete and uniform coverage of the chip surface.
4.5 The handheld device was sealed with sealing film, placed on the PCR adapter of the PCR instrument at 65° C. The lid of the PCR instrument was then closed for 30 minutes of reaction.
   5. cDNA Fragmentation
5.1 A PCR instrument was pre-set to 55° C. with the heated lid at 60° C., and a PCR adapter was placed to equilibrate at this temperature.
5.2 9 μL of TE buffer was added to 1 μL TME for 10-fold dilution, vortexed to mix thoroughly, and briefly centrifuged for use.
5.3 A TME fragmentation reaction mixture was prepared according to Table 3, vortexed to mix thoroughly, and briefly centrifuged for use. TMB and TME were both obtained from the Stereo-seq Library Preparation Kit, where TMB is the transposase reaction buffer, and TME is the transposase loaded with nucleic acid adapters.

TABLE 3
TME Fragmentation Reaction Mixture

| Component | Volume per reaction | |
|---|---|---|
| TMB | 20 | μL |
| 10-fold diluted TME | 2 | μL |
| Nuclease-free water | 78 | μL |
| Total | 100 | μL |

5.4 After completion of the second strand synthesis reaction, the handheld device from step 4 was removed from the PCR instrument. The device was gently tilted at an angle less than 20°, and the reaction mixture for synthesizing second strand cDNA was aspirated from one corner of the chip using a pipette.
5.5 The prepared TME fragmentation reaction mixture was mixed thoroughly, briefly centrifuged, and then added from one corner of the chip to ensure complete coverage of the chip surface.
5.6 The handheld device was sealed with sealing film, placed on the PCR adapter of the PCR instrument at 55° C. The lid of the PCR instrument was then closed for 10 minutes of reaction.
   6. Second Strand cDNA Elution
6.1 A PCR instrument was pre-set to 70° C. with the heated lid at 75° C., and a PCR adapter was placed to equilibrate at this temperature.
6.2 8 M KOH was diluted to 0.2 M using nuclease-free water, vortexed to mix thoroughly, and briefly centrifuged for use.
6.3 After completion of fragmentation reaction, the handheld device from step 5 was removed from the PCR instrument. The device was gently tilted at an angle less than 20°, and the fragmentation reaction mixture was aspirated from one corner of the chip using a pipette.
6.4 100 μL of 0.2 M KOH was added onto the chip to ensure complete coverage of the chip surface.
6.5 The handheld device was sealed with sealing film, placed on the PCR adapter of the PCR instrument at 70° C. The lid of the PCR instrument was then closed for 20 minutes of reaction.
6.6 After completion of reaction, the handheld device was removed from the PCR instrument. The device was gently tilted at an angle less than 20°, and the KOH solution was aspirated from one corner of the chip using a pipette and transferred into a 1.5 mL tube. 4 μL of 1 M Tris-HCl (pH 6.8) was added, and the solution was mixed thoroughly by pipetting. The solution was then divided into two PCR tubes for PCR reaction to obtain the PCR product.

### 7. PCR Amplification and Purification

7.1 The PCR Mix system was prepared according to Table 4.

TABLE 4
PCR Mix

| Component | Volume per reaction | |
|---|---|---|
| Second strand cDNA elution product | 50 | μL |
| PCR Amplification Mix | 60 | μL |
| PCR Barcode Primer Mix | 10 | μL |
| Total | 120 | μL |

7.2 The mixture was vortexed to mix thoroughly, briefly centrifuged, and placed into a PCR instrument for amplification according to the program in Table 5.

TABLE 5
PCR Amplification Program

| | | |
|---|---|---|
| Temperature | Time | Number of Cycles |

| Heated lid at 105° C. | on | | |
|---|---|---|---|
| 95° C. | 5 | min | 1 |
| 98° C. | 20 | s | 20 |
| 58° C. | 20 | s | |
| 72° C. | 30 | s | |
| 72° C. | 5 | min | 1 |

| | |
|---|---|
| 12° C. | Hold |

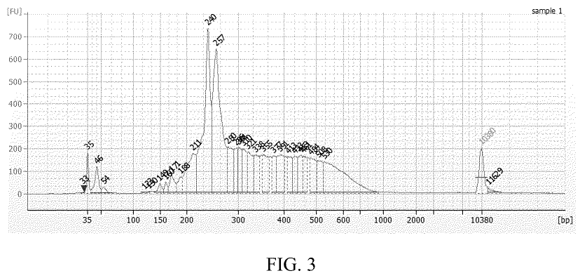

7.3 The PCR product obtained in step 6 was mixed with room-temperature equilibrated magnetic beads at a ratio of 1:0.8 (120 μL PCR product: 96 μL beads), vortexed to mix thoroughly, and incubated at room temperature for 5 minutes.
7.4 The tube was briefly centrifuged, placed on a magnetic rack, and allowed to stand for 3-5 minutes until the solution became clear; subsequently, the supernatant was carefully removed using a pipette and discarded.
7.5 The tube was kept on the magnetic rack, and 200 μL of freshly prepared 80% ethanol was added. The magnetic beads were rinsed by rotating the tube on the magnetic rack. After standing for 30 seconds, the supernatant was carefully removed and discarded.
7.6 Step 7.5 was repeated once.
7.7 Residual liquid in the tube was removed as much as possible. If a small amount remained on the tube wall, the tube was briefly centrifuged, placed on a magnetic rack for separation, and the remaining liquid was removed with a small-volume pipette.
7.8 The beads were air-dried at room temperature for 3-5 minutes until the bead surface appeared matte and without cracks.
7.9 20 μL of TE buffer was added, vortexed to mix thoroughly, and incubated at room temperature for 5 minutes. The tube was briefly centrifuged, placed on the magnetic rack, and allowed to stand for 3 minutes. Once the solution cleared, the supernatant was transferred to a 1.5 mL tube.
7.10 1 μL of the purified product was quantified using Qubit, followed by fragment analysis using the Bioanalyzer 2100. The quality control results of the sequencing library fragments are shown in FIG. 3, indicating that the majority of fragments were 200 bp to 300 bp in size.

### Comparative Example: Replacement of Bst Polymerase with Klenow Fragment

In this comparative example, Klenow fragment (BGI, 1000004293) was used to replace Bst polymerase for the second strand cDNA synthesis reaction. The procedure was as follows:
4.1 A PCR instrument was set to 37° C. with the heated lid at 42° C., and a PCR adapter was placed to equilibrate at this temperature. This PCR instrument was used for the subsequent step 4.5 reaction.
4.2 Near the completion of the RT reaction, a reaction mixture for synthesizing second strand cDNA was prepared according to Table 6, vortexed to mix thoroughly, and briefly centrifuged.

TABLE 6
Reaction Mixture for Synthesizing Second Strand cDNA

| Component | Volume per reaction | |
|---|---|---|
| 10x Klenow buffer | 10 | μL |
| 25 mM dNTP | 6 | μL |
| 0.1 μg/μL random primer | 10 | μL |
| 5 U/μL RNaseH | 1 | μL |
| Klenow fragment | 5 | μL |
| Water | 68 | μL |
| Total | 100 | μL |

All other steps were performed as described in the above Example.

### Result Analysis:

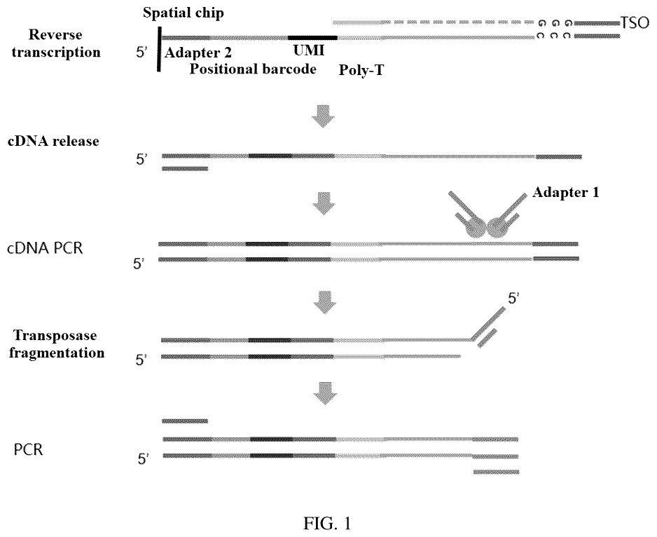

1. Sequencing libraries were prepared using the method of this Example (present method) and using the conventional spatial transcriptome method shown in FIG. 1 (original protocol). The comparison of the overall workflow time is shown below:

Present Method

| | | | Synthesizing | | Second | | | |
|---|---|---|---|---|---|---|---|---|
| | Reverse | Tissue | Second | Transposase | Strand | | | Total |
| Steps | Transcription | Removal | Strand | Fragmentation | Elution | PCR | Purification | Time |
| Reaction | 3 | 0.2 | 0.5 | 0.2 | 0.35 | 1 | 1 | 6.25 |
| Time (H) | | | | | | | | |

Original Protocol

| | Reverse | Tissue | cDNA | | | | Transposase | | | Total |
|---|---|---|---|---|---|---|---|---|---|---|
| Steps | Transcription | Removal | Release | Purification | PCR | Purification | Fragmentation | PCR | Purification | Time |
| Reaction | 3 | 0.2 | 3 | 0.5 | 1.3 | 0.5 | 0.2 | 0.75 | 1 | 10.45 |
| Time (H) | | | | | | | | | | |

This demonstrates that the method of the present disclosure can significantly shorten the sequencing library construction workflow and the time, thereby reducing costs.
2. Sequencing analysis showed that the sequencing library prepared using the method of the present Example had a duplication rate of 32.02%, whereas the sequencing library prepared using the conventional spatial transcriptome method (Specific construction method can be referred to the publicly disclosed method: Spatiotemporal transcriptomic atlas of mouse organogenesis using DNA nanoball-patterned arrays, https://www.cell.com/cell/pdf/S0092-8674 (22) 00399-3.pdf) had a duplication rate of 55.14%. Thus, the method of the present disclosure can significantly reduce duplicate sequences in the sequencing library.
3. The cDNA products obtained in Example and Comparative Example are shown in Table 7. It can be seen that, compared with Klenow fragment, using Bst polymerase for second strand cDNA synthesis resulted in higher cDNA concentration and total yield. Therefore, library construction using Bst polymerase achieves a better effect.

TABLE 7
cDNA Product Profile

| | Polymerase for | cDNA Product | Total cDNA |
|---|---|---|---|
| | Second Strand | Concentration | Product Yield |
| Groups | Synthesis | (ng/μL) | (ng) |
| Example | Bst polymerase | 6.37 | 191.1 |
| Comparative | Klenow fragment | 2.755 | 82.65 |
| Example | | | |

Reference in the specification to “one embodiment”, “some embodiments”, “example”, “particular examples”, or “some examples”, etc. means that a particular feature, structure, material, or characteristic described in connection with the embodiment or example is included in at least one embodiment or example of the present disclosure. In this specification, schematic depictions of the above terms do not have to be directed to the same embodiment or example. Furthermore, the particular features, structures, materials, or characteristics described may be combined in any suitable manner in any one or more embodiments or examples. Furthermore, combinations and sub-combinations of the various embodiments or examples and features of the various embodiments or examples described in this specification can be made by one skilled in the art without mutual contradiction.

While embodiments of the present disclosure have been shown and described, it is to be understood that the above-described embodiments are illustrative and are not to be construed as limiting the present disclosure. Changes, modifications, substitutions, and variations may be made in the above-described embodiments by those of ordinary skill in the art without departing from the scope of the present disclosure.

1. A method for constructing a sequencing library, the method comprising:
step A: subjecting mRNA to reverse transcription without adding a template switch oligo, thereby obtaining a reverse transcription product, wherein the 3′ end of the mRNA comprises a poly-A sequence, and wherein the mRNA is attached to a chip to which a probe comprising a poly-T sequence is attached by complementary pairing between the poly-A sequence at the 3′ end of the mRNA and the poly-T sequence on the chip;
step B: subjecting the reverse transcription product to fragmentation and addition of a first adaptor, thereby obtaining a fragmented adapter-ligated product, wherein the fragmented adapter-ligated product remains attached to the chip; and
step C: releasing the fragmented adapter-ligated product from the chip, thereby obtaining the sequencing library.

2. The method according to claim 1, wherein the reverse transcription product comprises a cDNA-mRNA hybrid strand, wherein the cDNA is a first strand cDNA.

3. The method according to claim 2, wherein step A further comprises, subsequent to the reverse transcription, synthesizing a second strand cDNA, wherein
said synthesizing the second strand cDNA is carried out using a random primer as a primer and using the first strand cDNA as a template.

4. The method according to claim 3, wherein said synthesizing the second strand cDNA is carried out using a polymerase, and the polymerase is at least one selected from Bst polymerase, Taq DNA polymerase, Klenow fragment, T4 DNA polymerase, DNA polymerase I, and reverse transcriptase having DNA-dependent polymerase activity, preferably, the polymerase is Bst polymerase.

5. The method according to claim 3, wherein said synthesizing the second strand cDNA is carried out under a condition of 65° C. to 70° C. for 20 to 60 minutes.

6. The method according to claim 1, wherein the probe is of a plurality of types, and each type of probe includes a unique positional barcode sequence, which has a one-to-one correspondence with the location of the type of probe on the chip.

7. The method according to claim 6, wherein the probe further comprises a second adapter sequence,
the 5′ end of the second adapter sequence is linked to the chip;
the 3′ end of the second adapter sequence is linked to the 5′ end of the positional barcode sequence; and
the 3′ end of the positional barcode sequence is linked to the poly-T sequence.

8. The method according to claim 7, wherein the probe further comprises a unique molecular identifier sequence, wherein:
the 5′ end of the second adapter sequence is linked to the chip;
the 3′ end of the second adapter sequence is linked to the 5′ end of the positional barcode sequence;
the 3′ end of the positional barcode sequence is linked to the 5′ end of the unique molecular identifier sequence; and
the 3′ end of the unique molecular identifier sequence is linked to the poly-T sequence.

9. The method according to claim 1, wherein the mRNA is derived from a tissue sample or a single-cell sample.

10. The method according to claim 9, further comprising, prior to step A:
contacting the tissue sample or single-cell sample with the chip; and
permeabilizing the tissue sample or single-cell sample to release mRNA from the tissue sample or single-cell sample.

11. The method according to claim 1, wherein in step B, the reverse transcription product is fragmented using a transposase or a fragmentase.

12. The method according to claim 11, wherein the transposase is Tn5 transposase.

13. The method according to claim 1, wherein in step C, the fragmented adapter-ligated product is released from the chip using an alkaline solution or formamide.

14. The method according to claim 13, wherein the alkaline solution is a strong alkaline solution, preferably KOH.

15. The method according to claim 1, wherein step C further comprises, subsequent to said releasing the fragmented adapter-ligated product from the chip, subjecting the fragmented adapter-ligated product to PCR amplification, thereby obtaining the sequencing library.

16. A method for sequencing transcriptome, the method comprising:
sequencing the sequencing library constructed by the method according to claim 1 to obtain sequence information of the sequencing library.

17. A method for sequencing spatial transcriptome, the method comprising:
constructing a sequencing library using the method according to claim 1;
sequencing the sequencing library; and
obtaining spatial transcriptome information of a sample to be detected based on the sequencing results.

18. A method for sequencing transcriptome, the method comprising
sequencing the sequencing library constructed by the method according to claim 6 to obtain sequence information of the sequencing library.

19. A method for sequencing spatial transcriptome, the method comprising:
constructing a sequencing library using the method according to claim 6;
sequencing the sequencing library; and
obtaining spatial transcriptome information of a sample to be detected based on the sequencing results.

20. A method for sequencing spatial transcriptome, the method comprising:
constructing a sequencing library using the method according to claim 8;
sequencing the sequencing library; and
obtaining spatial transcriptome information of a sample to be detected based on the sequencing results.
